(19) United States
(12) Patent Application Publication
(10) Pub. No.: US 2022/0208332 A1
(43) Pub. Date: Jun. 30, 2022

(54) INTERACTIVE HUMAN ACTIVITY TRACKING SYSTEM

(71) Applicant: BRICKFIT PTY LTD., Victoria (AU)

(72) Inventors: James B. Kelly, Victoria (AU); Michael A. Jones, Victoria (AU); Ryan N. McNaught, Victoria (AU)

(73) Assignee: BRICKFIT PTY LTD, Victoria (AU)

(21) Appl. No.: 17/609,967

(22) PCT Filed: May 11, 2020
(86) PCT No.: PCT/AU2020/000039

§ 371 Date: Nov. 9, 2021

(30) Foreign Application Priority Data
May 10, 2019 (AU) 2019901607

(51) Int. Cl. G16H 20/30; G06F 1/16; A61B 5/11; A61B 5/00; A63H 33/04; A63B 71/06; H04L 67/306; H04W 4/38

(52) U.S. Cl. CPC G16H 20/30; A63B 2230/75; A61B 5/1118; A61B 5/681; A61B 5/7475; A61B 5/0022; A63H 33/04; A63B 71/0622; H04L 67/306; H04W 4/38; A61B 2503/06; A63B 2071/0663; A63B 2208/12; A63B 2220/20; A63B 2220/62; A63B 2225/50; A63B 2230/06; G06F 1/163

(57) ABSTRACT

A system for directly tracking human activity is described. The system comprises a plurality of interactive activity objects such as, construction toys, that interact with each other in one or more activities involving physical manipulation of the interactive activity objects by a person; at least one wearable device associated with the person to track the person's activity by sensing the person's movements, to obtain data directly relating to the person's activity; and a database to record the person's direct activity and at least some of the person's direct physical manipulation of with the interactive activity objects.

### FIELD OF THE INVENTION

The present invention relates to a human activity tracking system. The invention has particular application to monitoring a person's physical activity and incentivising them to perform interactive tasks or games, particularly with construction toys. However, the invention also has broader application to other types of activities.

### BACKGROUND TO THE INVENTION

Childhood obesity, and obesity generally, is a major problem throughout the world, particularly in developed countries. One contributing factor to this is a sedentary lifestyle. The influence of technology means that many children and adults are spending more time than ever engaging in sedentary behaviour, due to excessive use of electronic media. About four out of five Australian children do not get the hour or more of physical activity they require each day. Also, fewer than 50% of Australian adults meet the physical activity guideline of 150 to 300 minutes a week and up to 60% are overweight or obese.

Adolescent obesity is also becoming a major problem worldwide, and is related to a range of adverse health effects in adulthood.

Currently, many children's toys and products are targeted at users who are expected to spend large amounts of time indoors, indulging in activities that do not require physical exercise. Games that use technology promote a sedentary lifestyle, which in turn contributes to the problems of childhood and adolescent obesity.

Modern parents also tend to have busy schedules of their own, which mean they can struggle to stay involved and monitor their children's activities on a day-to-day or moment-to-moment basis.

European Patent application No. 3219369 to FUJITSU LIMITED discloses a construction toy which includes numerous inter-locking blocks which have controllable connectors that are adjustable between at least two connectivity states so as to allow, encourage, discourage or prevent connection of the construction toy to another construction toy. The connectivity states may for example include: male, female and neutral states or enabled and disabled. Each construction toy may have a location sensor. A host computer may monitor construction of a set of the construction toys and determine transformations to be performed on the connectors and the individual blocks may detect and send location data to the host computer and execute received commands. The computational monitoring includes recording a player's interactions with the blocks and analysing player behaviour to determine aspects such as, behaviour variability, complexity level and possible detection of autism.

WO 2016/055862, the publication of International Patent Application PCT/IB2015/002127, to DXTR Tactile IvS teaches another construction toy made up of a set of interactive objects, each with a processor and a sensor, which communicate with each other and a base station or client device and which can be coupled together to allow the creation of various three dimensional shapes. Player behaviour such as, manual ability and dexterity; cognitive ability; creativity; memory and retention can be determined and challenges may be provided based on past performance.

It is a preferred object of embodiments of the present invention to address or at least ameliorate one or more of the aforementioned problems of the prior art and/or provide a useful commercial alternative.

The reference to any prior art in this specification is not, and should not be taken as, an acknowledgement or any form of suggestion that the prior art forms part of the common general knowledge.

### SUMMARY OF THE INVENTION

In one form, although it need not be the only or indeed the broadest form, the invention resides in a system for directly tracking human activity comprising:

a plurality of interactive activity objects that interact with each other in one or more activities involving physical manipulation of the interactive activity objects by a person;

at least one monitoring device associated with the person, to obtain data directly relating to the person's activity; and

a database to record the person's direct activity and at least some of the person's direct physical manipulation of the interactive activity objects.

The interactive activity objects are preferably objects that are used for active play (as opposed to the sedentary play experienced with most electronic devices). Accordingly, the present invention can be used to bridge the gap between electronic devices and physical play. Rather than typical uses of technology, which add to the sedentary lifestyle of many children, the present invention can be utilised to promote physical activity. By integrating aspects of digital and physical play, the present invention may be used to combat ongoing issues such as adult, child and adolescent obesity problems.

In some embodiments, the objects may be construction toys such as blocks or “bricks,” and may include connecting components to connect the objects together when performing the activity. For example, the interactive activity objects may comprise construction bricks as commonly sold under the “Lego” trade mark, although different interactive activity objects may be used in different embodiments of the invention. The invention could be used for a wide variety of toys, including model kits or robot kits. Suitable toys are commonly sold under trade marks such as “Duplo”, “Meccano, “LaQ” or “IQKey” (formerly known as “Capsela”).

The database may record data relating to multiple persons. The person(s) may be child(ren); adolescents or adults. The data may comprise one or more of: user data; activity data; and assembly data. The user data may include details of a person's age, sex, location, cognitive level or various other hobbies or interests of the person. The user data may further comprise components of one or more sets of activity objects associated with the one or more person. The activity data may comprise information regarding the amount of activity performed by the person such as, physical manipulation of the interactive activity objects; steps; cadence; distance; energy expended; sleep data; and heart rate tracking. The activity data may further comprise one or more physical activity challenges which the person may nominate and may be ticked off upon completion. The activity challenge may be ticked off by a parent or guardian. The assembly data may comprise details of one or more assemblies constructed with the activity objects; a duration of time the activity objects were interacted with; a quantity of movement performed, and/or a measure of the energy (calories) expended by the person when using the activity objects. The activity data may also comprise data for other activities independent of the activity objects—for example, community activities such as fun runs or triathlons. GPS data may be used to indicate and/or verify that these community activities are performed.

The at least one monitoring device may comprise a wearable device. The wearable device may be worn about the wrist, and may comprise a watch or smart watch. The wearable device may track the user activity by sensing the user's movements, or may receive input from the person using a control interface on the wearable device. The control interface may comprise a display (which may be a touch screen), and/or buttons to enter data. The monitoring device may comprise one or more sensor and/or receiver such as, one or more accelerometer, one or more gyroscope and a GPS receiver. The device may provide an alert when it has detected a person may be using the activity objects. The alert may comprise an audible or haptic alert. A user may provide a confirmation that the activity objects are being used in response to the alert.

The at least one monitoring device may comprise a mobile phone or other mobile computing device such as a tablet computer. The tablet computer may comprise a display (which may be a touch screen). A computer program such as an app may be installed on the mobile phone to provide a control interface for the user to interact with the app, and to receive the data relating to the person's activity. The computer program may communicate with a remote computer, such as a server, which stores the database. The communication may be to upload the data from the mobile computing device to the database, and/or download information to the mobile computing device regarding the person, the activities completed by the person or new activities for the person to complete, or other associated information.

In a second aspect of the invention, there is provided a computer-implemented method of directly tracking human activity comprising:

obtaining data regarding a person's direct activity with a plurality of interactive activity objects that interact with each other in one or more activities involving physical manipulation of the interactive activity objects by a person; and

uploading the data to a database.

According to the second aspect, the data may be obtained by sensing the person's movements with the least one wearable device associated with the person, the wearable device tracking the person's activity by sensing the person's movements, to obtain data directly relating to the person's activity.

The second aspect may further comprise uploading to the database the data comprising the person's direct activity and at least some of the person's direct physical manipulation of the interactive activity objects.

The method may further comprise receiving data from a third party server, such as a social networking server to provide information relating to a third party social network such as Strava, Facebook or GameCenter.

In another aspect of the invention, there is provided a mobile computing device for tracking interactive activity of a person, comprising:

at least one processor; and

a memory in communication with the processor, the memory storing instructions that, when executed by the at least one processor, cause the platform to perform the method of the second aspect of the invention.

The mobile computing device may comprise a mobile phone, or a wearable device.

In a third aspect of the invention, there is provided a system for directly tracking human activity comprising:

a plurality of interactive activity objects that interact with each other in one or more activities involving physical manipulation of the interactive activity objects by a person;

monitoring means for obtaining data relating to the person's activity; and

a database to record the person's activity and at least some of the person's physical manipulation of the interactive activity objects.

According to the third aspect, the data may be obtained by sensing the person's movements with the least one wearable device associated with the person, the wearable device tracking the person's activity by sensing the person's movements, to obtain data directly relating to the person's activity.

The third aspect may further comprise uploading to the database the data comprising the person's direct activity and at least some of the person's direct physical manipulation of the interactive activity objects.

The interactive activity objects may be comprised in a kit. The kit may comprise instructions for a set of assemblies, or configurations, of the activity objects. One or more additional instructions and/or add-on kits for further configurations may be provided, if a goal is achieved. The goal may comprise an activity goal, or a kit assembly goal, or both. The activity goal may comprise a duration and/or frequency of play. The kit assembly goal may comprise a pre-determined assembly of the plurality of interactive activity objections such as a goal structure for example in the form of a dwelling or mode of transport. Achievement of a goal may require verification, which may be provided by a parent or guardian, or by uploading a photograph of the completed assembly configuration, and/or by artificial intelligence to analyse the person's movements and/or the photographs provided and verify that they have achieved a particular goal. The photograph of the completed assembly may be received be systems of the first or third aspects and analysed to verify that the goal has been achieved. The method of the second aspect may further comprise receiving a photograph of the completed assembly configuration. Achievement of a goal may enable various rewards, including badges, privileges on the platform, or access to exclusive kits or activity objects that are not available elsewhere.

One or more leader board may rank the person. The ranking may be for one or more particular activity.

Further aspects and/or features of the present invention will become apparent from the following detailed description.

### BRIEF DESCRIPTION OF THE DRAWINGS

In order that the invention may be readily understood and put into practical effect, reference will now be made to embodiments of the present invention with reference to the accompanying drawings, wherein like reference numbers refer to identical elements. The drawings are provided by way of example only, wherein:

FIG. 1 is a system diagram depicting a system of tracking interactive activity of a person, according to an embodiment of the present invention.

FIGS. 2A and 2B are schematic diagrams showing an exemplary computer system for use in embodiments of the present invention.

FIGS. 3A to 3Q are representations of a Graphical User Interface (GUI) according to one embodiment of the invention.

Skilled addressees will appreciate that elements in the drawings are illustrated for simplicity and clarity and have not necessarily been drawn to scale. For example, the relative dimensions of some elements in the drawings may be distorted to help improve understanding of embodiments of the present invention.

### DETAILED DESCRIPTION OF THE INVENTION

As used herein “interactive” is used to mean acting one upon or with the other.

The “interactive activity objects” may be fit together into an assembly in the shape, form or construction of a goal structure. One set of interactive activity objections may be fit together into multiple different goal structures. The supplementation of the set with additional interactive activity objects may increase the number of different goal structures which may be constructed.

As used herein “planar” when used in reference to the interactive activity objects means flat or substantially flat such as, LaQ parts.

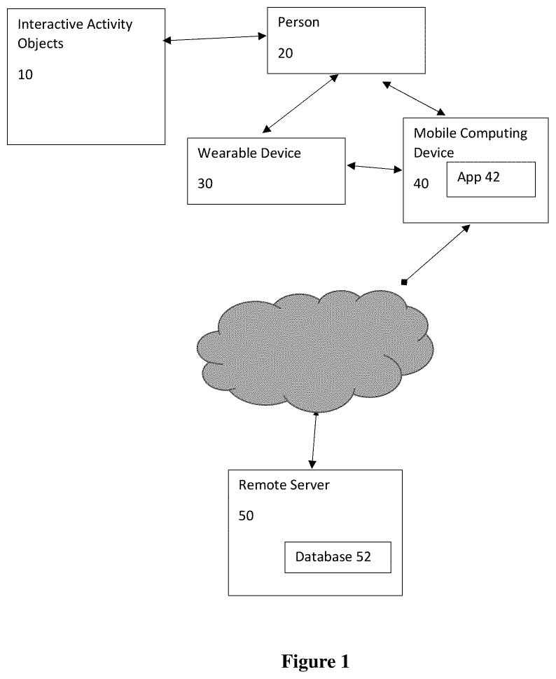

FIG. 1 depicts an exemplary system according to an embodiment of the present invention. The system includes a plurality of interactive activity objects 10, which a person 20 may interact with. The person 20 may be a child, and in this embodiment they wear a wearable device 30, which is able to collect information associated with the person 20 and their interactions with the interactive activity objects 10.

The wearable device 30 shown in FIG. 1 is connected to a mobile computing device 40 in the vicinity of the person—for example, via a WiFi, BlueTooth or other data connection. The mobile computing device 40 may store the data from the wearable device 30 locally, in an internal database (not shown in the embodiment of FIG. 1), which can be viewed on the mobile computing device 40 itself. Alternatively, or additionally, the mobile computing device 40 may upload the data (via a network such as the Internet) to a database 52 on a remote server 50.

In either case, the mobile computing device 40 may have an app 42 installed on it, to enable various functions (to be described later) including to allow a user to view the data collected about the person's 20 interaction with the interactive activity objects 10.

In a preferred embodiment, the interactive activity objects 10 are toys that a person can use to complete particular activities. They are preferably toys requiring active play or physical play—for example, the activity objects 10 may be construction bricks or sets that a person 20 can physically interact with. The objects 10 may mechanically interact with each other, for example by having connection mechanisms to enable multiple objects to be connected together. Further, the objects may be square, rectangular, triangular or custom-shaped bricks, blocks, rods, strips, plates, girders, wheels, axels, gears, and may be assembled together into different assembled configurations. The interactive activity objects 10 may be any combination of planar and/or non-planar objects. One simple example of an interactive activity object is a construction brick sold under the “Lego” trade mark. These bricks comprise a variety of different shapes, and may be assembled and/or connected together in a variety of different configurations.

An assembled configuration of objects 10 may be associated with a particular “task” to be completed by the person 20. The person 20 may be prompted to complete tasks, each of which involve assembling the bricks into a particular configuration.

Of course, in other embodiments, different types of activity objects 10 may be used. In some embodiments, the activity objects 10 may be model construction sets or robot construction sets, that the person 20 can assemble. In yet other embodiments, the activity objects 10 could be other interactive toy components, such as jigsaw pieces.

The wearable device 30 may be a watch or similar device such as an activity monitor worn on the wrist of the person 20. The wearable device 30 can monitor the activity patterns of the person 20 (e.g. monitor the amount of movement of the person's 20 wrist). In different embodiments, this may enable the wearable device 30 to determine when the person 20 is engaged in active play (as opposed to the sedentary play experienced with most electronic devices), and further to determine how much movement the person 20 is performing.

The device 30 may comprise one or more sensor and/or receiver such as, one or more accelerometer, one or more gyroscope and a GPS receiver. The device 30 may provide an alert when it has detected a person may be using the activity objects. The alert may comprise an audible or haptic alert. A user may provide a confirmation that the activity objects are being used in response to the alert.

In accordance with this embodiment of the invention, the system utilises a database 52 to store and access information. In particular, in this embodiment, the types of information stored in the database include user data (including user profiles); activity data; and assembly data. Further details of the types of information stored in the database 52 are provided below.

In particular, the platform may create a profile for each person whose data is stored on the system—different persons can be identified by unique identifiers such as. identification numbers, usernames or actual names with associated email addresses. A parent or guardian may manage a profile for each child in their care. Profiles may be password protected to control access to the data stored in the database 52.

The specific types of user data stored in the database 52 will depend on privacy considerations, that may require the consent of the person 20 who is the subject of the profile, or the consent of their parent or guardian. Each user profile may include the age of the person 20, the gender/sex of the person 20, and details of their location (e.g. their state and country) and their school grade. In some embodiments, more specific information may optionally be provided and tracked, including data relating to the health of the person—such as height, weight, body fat percentage, waist circumference, body mass index (BMI), resting heart rate or any other measure that is consented to and considered relevant for a particular embodiment.

User data may also include the user's hobbies or interests, which may be input by the person (or, for a child, may also be input by a parent or guardian), or may be at least partially determined based on the types of activity objects 10 used or kits purchased. This information may be used to suggest appropriate kits or assembly configurations of activity objects 10 for the person. Access may be provided to specific kits (which may be mailed to a person if certain achievements are met or tasks completed), or new assembly instructions may be provided electronically for new configurations for a kit of activity objects 10 already owned by the person.

A cognitive level or achievement level may also be stored in the user profiles in the database, which may be initially set based on their age, but may be updated based on the difficulty of tasks completed and achievements unlocked, as the person's or child's ability improves. The cognitive level or achievement level may also be used to select or suggest assembly configurations for the person 20 to attempt next, with the activity objects 10.

Activity data stored in the database 52 may comprise information regarding the amount of activity performed by the person such as, steps; cadence; distance; energy expended; sleep data; and heart rate tracking. The activity data may also comprise
the duration for which the person has performed activities with the interactive activity objects 10. This information may be collected by having the person 20 click a start and stop button (at the start and end of a particular activity) on the wearable device 30 or using the app 42 on the mobile computing device 40. The information may also be collected by responding to an alert provided by the wearable device 30 when it has detected the person may be using the activity objects. The alert may comprise an audible or haptic alert. In response to the alert, the user may provide a confirmation that the activity objects are being used. Alternatively, the person's movements may be tracked using the wearable device 30 to measure and/or monitor when the person 20 is moving in a particular way associated with them interacting with the activity objects 10. Software on the wearable device 30 or mobile computing device 40 may automatically detect when the person 20 is moving in this way.
the amount of movement performed by the person when performing activities. This may be tracked (or approximated) using tracking information obtained from the wearable device
the amount of energy (e.g. an estimation of calories) used by the person 20 when performing activities. This may be a calculated based on the amount of movement measured for the person 20, in combination with user data such as the weight of the person 20.
GPS data to partner with events such as a park run, or other fun runs, bike rides, triathlons or community activities. Completion of these community activities may unlock specific achievements, and/or may unlock exclusive kits associated with those community activities.
one or more physical activity challenges may be provided in the app which the person can nominate and a parent or guardian may tick off upon completion.

The activity data may be broken down into tasks completed by a person 20. For example, the activity data may contain information about the duration of time and amount of movement required to assemble a particular configuration of “Lego” bricks, or (in other embodiments, to assemble a particular model or robot. For example, the activity objects 10 may be sold as a kit that can be assembled into a designated shape (such as a dinosaur, a car, a castle), and assembly of the shape constitutes a particular task. Each kit of activity objects 10 may be assembled in multiple possible configurations, and each configuration (or some designated configurations) may constitute a separate “task” that a person 20 can complete. Upon purchasing a kit, a person may be provided with a code to enter into the app, specific to the task, so that their activity with the activity objects 10 can be associated with that task.

When a person starts a particular task, they may click a “start” button on either the wearable device 30 or the mobile app 42. Similarly, when completing a task, they may click a “stop” button. The total time taken by the person 200 may be calculated based on these start and stop times.

In between the start and stop time, the wearable device 30 may monitor the amount of movement performed by the person to finish the task. The amount of movement may be measured in any of various units—such as total centimetres moved by the wearable device 30, total acceleration experienced by the wearable device 30, or total number of individual movements made by the person.

The completion of a particular task may be associated with different achievements or levels within the app 42. For the purposes of this specification, “achievements” are considered to be any reward associated with performance of activities with the activity objects 10—for example, a user may be provided with extra functions within the app 42, or they may have extra features added to their profile in the database 52, such as badges, trophies, photos or other rewards. They may be enabled to purchase new activity objects 10 from an online store available through the app 42, or obtain new assembly instructions for a kit of activity objects 10 they have already bought.

Achievements may be unlocked for performing a certain quantity of activities, and/or for completing tasks. For example, achievements may be unlocked for spending 1 hour, 5 hours, 10 hours or some other time threshold using the interactive activity objects 10. Achievements may also be unlocked for making a certain amount of movement with the blocks, or for completing designated tasks of specified difficulty levels. Achievements may also be unlocked by completion of one or more physical activity challenges which the person may nominate and may be ticked off, for example by a parent or guardian, upon completion.

The verification may be provided by a parent or guardian, or by uploading a photograph of the completed assembly configuration, and/or by artificial intelligence to analyse the person's movements and/or the photographs provided and verify that they have achieved a particular goal.

The activity goal may comprise a duration and/or frequency of play. The kit assembly goal may comprise a pre-determined assembly of the plurality of interactive activity objections such as a goal structure for example in the form of a dwelling or mode of transport.

In this way, the invention can be used to encourage people and children to use the activity objects 10, to create a sense of fun, achievement, excitement and/or competition, and thereby promote increased physical activity.

In some cases, competitions may be held for completing a task in a certain time or by a certain date. A person 20 may be required to verify that they performed the task by taking a video of themselves performing the task, or by taking dated photos of themselves performing the task. This may be cross-checked using movement information from the wearable device 30, to ensure that the movements were within expected bounds, and with evidence of the end result to verify that it meets the assembly instructions for that task.

The user profiles in the database 52 may include a “friends” list for each person 20. Each person 20 may be able to search for connect with other uses using a typical procedure of friend requests and acceptances, to build a social or “friend network” using the invention. Alternatively, this friend network may be built by linking to existing social networks such as Strava, Facebook or GameCenter.

Each person may be enabled to share different achievements that they have unlocked with their friend network, or share photographs of constructions they have made using the activity objects 10. This further incentivises persons 20 to use the activity objects 10, and to engage in physical activity/play.

The system may also monitor the activity of the person 20, and generate alerts or notifications when certain conditions are met—for example, when an achievement is unlocked; when a target amount of use is achieved; when a task of a new ‘level’ of difficulty is achieved; or when a target amount of use is not achieved (for example, a parent may specify that they would like their child to play with the objects 10 for a certain amount of time each day). Notifications may be sent to multiple devices associated with different people—for example, notifications may be sent to child's own device, the parents' devices, and/or an authorised carer or teacher who may be working with the person or child 20.

The assembly data may comprise details of one or more assemblies constructed with the activity objects 10; a duration of time the activity objects 10 were interacted with; a quantity of movement performed, and/or a measure of the energy (calories) expended by the person when using the activity objections.

In addition to the above features that give benefit to users of the activity objects 10 (or their parents or guardians), the present invention can provide benefits to manufacturers of the activity objects 10. Specifically, the activity data can give insight into the way that people are using the activity objects, and the difficulty of different tasks. For example, the activity data stored in the database 52 may be used to determine how children of a particular age fare when attempting to perform a particular task. For example, the data may indicate that a task of building particular configuration of bricks is too difficult for children of a particular age (that is, it takes too long, or those children rarely finish it) or too easy (the children finish it too quickly). It may also give insight into the average amount of time or amount of movement required to build particular configurations of the activity objects 10. The information may be used as a guide when selling or marketing the activity objects 10, or may be fed back into the design stage of the activity objects 10.

This information, regarding average performance for a particular demographic, can then be used to give further insight regarding individual children. For example, it may be determined from the data that a given child moves more or less than average when playing with the activity objects 10, is faster or slower than average at completing tasks, or has a lower completion rate for actually finishing tasks (e.g. they may leave some constructions unfinished). It may also be determined that a particular child spends longer or shorter amounts of time in individual sessions with the activity objects 10, suggesting longer or shorter attention spans. This may be useful in identify, for example whether a particular person 20 has developmental difficulties or aptitudes.

One or more leader board may also be maintained. The leader board may rank the person 20 among a plurality of other users with similar devices 30. The ranking may be for one or more particular activity.

Although the embodiment of the present invention shown in FIG. 1 includes three electronic computing devices (two monitoring devices—being the wearable device 30 and mobile computing device 40—and a remote server 50), in different embodiments, different combinations of computing devices may be used. For example, the wearable device 30 may communicate directly with the remote server 50 in some embodiments, without the need for a mobile device 40 such as a mobile phone. Alternatively, in some embodiments, the wearable device 30 may not be present, and a subset of information may be gathered simply using the mobile phone (e.g. if the person presses a start and stop button to simply measure the amount of time taken to perform a designated task).

The data may be obtained, stored, and tracked, using a variety of devices, including mobile computing devices such as tablets, laptops or mobile phones, or alternatively using other devices such as desktop computers.

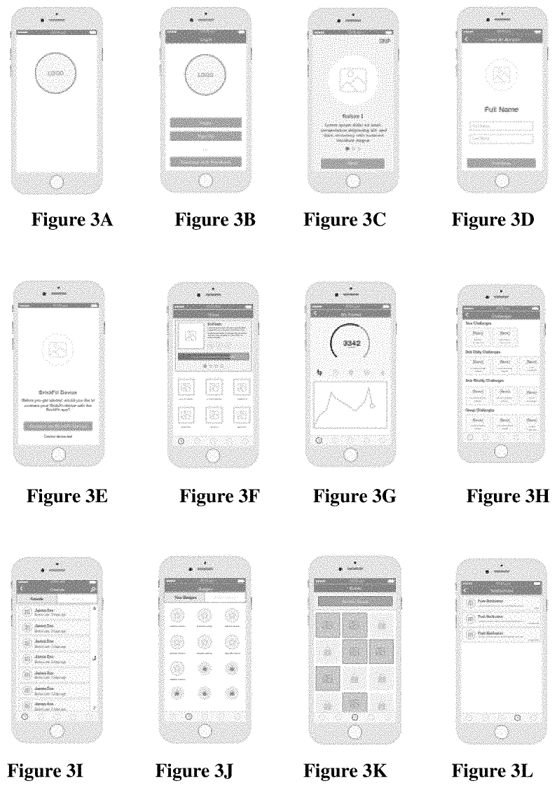

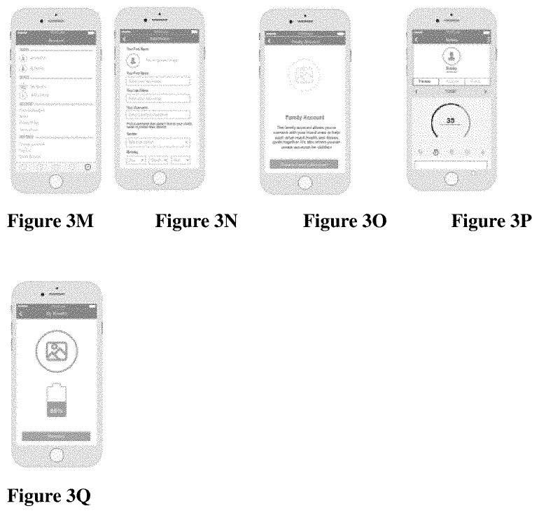

FIGS. 3A to 3Q show representations of one embodiment of a Graphical User Interface (GUI) that may be displayed on a mobile computing device 40. FIG. 3A shows a splash or launch screen. FIG. 3B shoes an example login and/or sign up page. FIG. 3C shows an initial screen of an onboarding slideshow according to one embodiment of the invention. The slideshow may explain how to use the app 42 and/or wearable device 30 and optionally also some of the benefits of their use.

FIG. 3D shows an example screen as part of a create account set up. A user may be required to enter their full name including their first and last names before progressing to additional, respective screens for entry of one or more of an email address; entry of a password; settings such as allowing push notifications.

FIG. 3E shows an example screen as part of the GUI for app 42 to set up wearable device 30 with the app 42 and mobile computing device 40. The set up may use Bluetooth. The user may choose to set up and thereby connect wearable device 30 or skip this process. The set up may comprise an acceptance of terms and conditions. The set up may comprise a search for a compatible wearable device 30. The user may select a desired wearable device 30 from those found. Once the user connects the wearable device 30 with the app 42, a connected device welcoming screen may appear along with a button to start.

FIG. 3F shows an example home screen which may provide access to all the functions required to navigate app 42. The home screen may comprise a slider indicating the builds they are aiming to unlock in a carousel slider. Below this are options to view a user's fitness levels; challenges; friends; badges; builds; and groups they are part of.

FIG. 3G shows an example fitness screen showing steps. There may be other similar screens showing for example, activity minutes, distance, heart rate tracking and/or energy expended. The data for several days, weeks or months may be viewed and compared. A user may select different screens showing the different parameters by for example, swiping a touchscreen.

FIG. 3H shows an example Challenge screen which allows a user to view all the challenges they can participate in and/or are participating in. When viewing challenges, a user may be presented with four columns such as, “Your Challenges”; “Solo Daily Challenges”; Solo Weekly Challenges”; and “Group Challenges”. Each Challenge may be created and set in an Administration panel of the App 42. When a user selects a solo challenge, the may be presented with the name of the challenge, the type of challenge, the description and a Start Challenge button which when selected, activates the challenge for the user. When a user selects a group challenge, they are presented with the name of the challenge, the type of challenge, the description and a “Select Member button”, which when selected directs users to a screen to nominate the challenge. A user can create a new group by creating a new name and adding members or tapping on an existing group and nominating the challenge to the group. The user can search for other members they have added as friends. Each selected member may receive an invitation they must accept to join. When viewing a Group Challenge, the user can view the name of a challenge, the requirements to complete it and the members who are participating. There may be an option to leave a challenge. If a Group Challenge is a competition which involves users having to participate in a “race”, a leader board may show a current ranking.

FIG. 3I shows an example View Friends screen according to one embodiment of the GUI. Advantageously, users can view all the friends and groups they are part of in one place. This gives each user access to viewing their friend's badges and builds. Each user also has the ability to create their own groups. Each user can view all their friends they have made part of their network, these may be displayed in alphabetical order. The user can use a slider on the right to scroll between names. There may also be the ability to switch between Friends and Groups. A search icon may be displayed in for example the top-right hand corner of the GUI. Each user can search for other users on the platform. When searching a name, users that are have followed as well as users that may be added may be shown. By tapping on a name, a user's profile may be viewed. When viewing another user's profile, that other user's badges and builds they have unlocked may be viewed. There may also be an “Add Friend” button which may send a friend request to the other user to accept. When viewing a user's profile that is already followed, the user's badges and builds may also be viewed. An option to remove a friend from a network may be comprised for example with a button displayed on the screen. The user ay be able to view each of the groups they have been added to. The groups that the user has created and the administration of may appear with an edit button in the top right-hand corner. There may also be the ability to create groups. When selecting the members that are going to be part of the group, each user may select from their list of friends. There may be a search icon displayed on the screen and a create group button also displayed on screen. When viewing a group that a user has administrator access to, the user can view how many ongoing, completed and expired challenges the group has participated in. The administrator users can also delete the group, add and/or remove members and create challenges. The administrator user can also view all the members that had accepted and joined. When viewing a group that a user has been invited to, the invited user will be presented with the same functionality as a group they have administrator access to, without being able to delete a group and add and/or remove members.

FIG. 3J shows one embodiment of a Badges screen as part of the GUI. Users may view all the badges they have achieved from completing challenges. When a user views their badges, they may be presented with an option to switch between their individual badges and group badges. Each user may view unlocked and locked badges. Locked badges may present a lock icon and the badges unlocked may display with their name. This layout may display the same for the group badges. When a user selects an unlocked badge, the user may view the name of the badge, the requirements it had taken to achieve, the awarded date and the requirements to unlock the next badge. If the user selects a badge that is still locked, the user can not view the name of the badge, they will only be presented with the requirements to unlock the badge and a lock icon.

FIG. 3K shows the GUI displaying one embodiment of a Build screen which allows a user to view all the builds that can be unlocked. When a user unlocks a build, they may be able to access the build instructions. If the build is still locked and the user taps on one of them, a pop-up may appear explaining that it is locked and the requirements needed to unlock it. Viewing an unlocked build allows the user the option to view the instructions. The badges required to unlock the build may also be displayed. The user may also be presented with an image of what the build will look like. The user also have the ability to take an image of the build and upload it to the Build screen. This upload may only appear on the individual user's Build screen. Once the user has uploaded an image of their build, the layout may change and will view full screen. Each image uploaded may display as a carousel slider. The carousel slider may show the date the build was unlocked, the name, badge requirements and a button to view the instructions. When viewing the instructions, a user may view them in landscape and in a .pdf view.

FIG. 3L shows one embodiment of a Notification screen of the GUI. Users may receive push notifications when certain activity in the app 411 affects them. Each user may receive different notifications comprising one or more of: Friends Request; Unlocking Badges; Unlocking Builds; Completed Challenges; Group Requests; Child Account; Group Requests; Child Build Unlocks; Child Completed; Challenges Unlocking Badge; and Child Unlocking Badge.

FIG. 3M shows one embodiment of a Accounts screen of the GIU. Each user may have access to editing their profile, the settings of the application and the ability to add devices and family members. When a user views the account settings, they may be presented with access to one or more of profile; My Family; their BrickFit Device; Push Notifications; About information screen; Privacy Policy; Terms of Use; Change Password; Log Out; and Delete Account. To change a user password, the user may be required to type their old password in before typing their new password. When creating the new password, the user may need to confirm and verify their decision before continuing.

FIG. 3N shows an example Profile screen according to one embodiment of the GUI. Users may edit their profile information which includes their name; username; gender; and date of birth. When editing a profile, a user may have access to changing one or more their profile image; First name; Last name; Username; Gender; and Birthday. There may be a save button below these fields to confirm your decision.

An example Family Account screen of the GUI is shown in FIG. 3O. Users may monitor their children's accounts through the app 42. This may allow each user visibility over everything their child is doing in the app 42. If a user selects the My Family option for the first time, they may be presented with a message explaining to the user what the family account is and the option to create one. When viewing My Family after proceeding through the previous screen, the user will be presented with their account name as the account holder of the family. There may be a create icon on displayed on the screen which allows the user to create an account for a child. There may also be an optional icon on the screen that allows the user to shut down the family account. To add a child to a family account, the user must type their password in to confirm their decision. This is also to confirm that the user creating the account is the account holder of the user using the device. The user may then be presented with a message explaining and reminding the user that the data stored on the app 42 is important and that they are transparent on how the data is going to be used. The user may be presented with a Privacy Notice they must agree to before continuing. To add a child, the user may only provide app 42 with one or more of child's first name; a username; gender; birth date; and height. Other users can not view this information. The other users can only view the username. There may be a Create Account button at the bottom of the screen. At this stage, the user may the ability to pair the child's wearable device 30 with their mobile device 40. The use may also have the option to skip this process and to do it later. A Prepare screen may be provided to prepare the user in setting up their child's account. Every time the user selects “next”, each point may be ticked off and the user may be moved onto the next stage. Once the user has proceeded through the stages, they may be presented with a “Let's get started!” button. Once a child is added to the My Family account, they may be listed under the account holder's name. The user may add or remove a child from the list. When viewing a family member's profile, the user may be able to view one or more of the child's fitness achievements; badges; and builds unlocked. The user may also have the ability to switch the child's account. When the user scrolls down the page, the user's profile image; name; and user name; may shrink and slide to one side of the screen. This then gives the user more real estate to view more information. This concept may apply for Fitness; Badges; and Builds.

A Family Account may also track and record “family interactions” such as, family challenges and/or specific activities achieved. The family challenges may include one or more of most steps; most time at high intensities of movement; most regular to conduct activity; and longest activity streak.

FIG. 3P shows one example of a Family member account screen of the GUI according to one embodiment of the invention. A child's account can switch back to a parents account by entering their password. For a parent to switch from a child's account to a parent's account, they may select the options icon displayed on the GUI select the “Switch to Parent Control” option. This may then require the user to place their password into the app for them to switch. To switch to a parent's account, the user may enter the parent's password into app 42.

FIG. 3Q shows one embodiment of a Battery status screen of the GUI. A user may view the device's battery level to ensure their wearable device 30 is sufficiently charged. The user may have access to the device's battery levels. When the battery drops to 10%, the user may receive a notification reminding them they must charge the wearable device 30. There may also be a “Remove” button at the bottom of the screen.

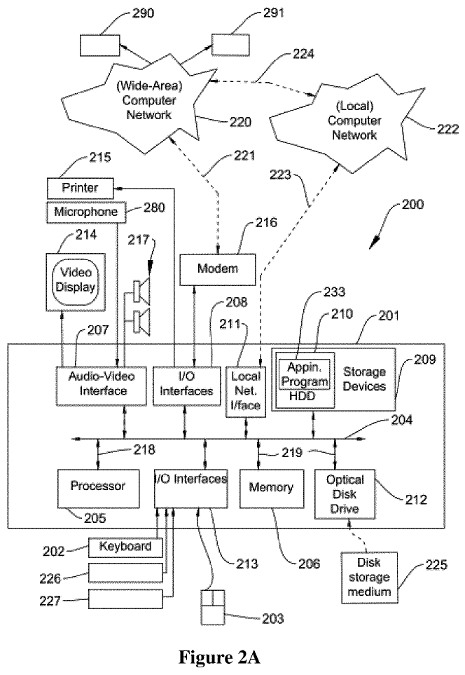

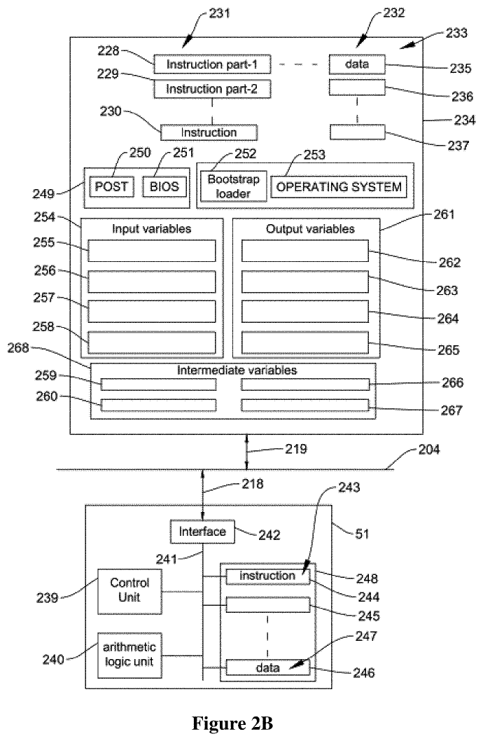

One embodiment of a computing device suitable for use in the present invention is shown in FIGS. 2A and 2B. In the embodiment shown, computing device 200 comprises a computer module 201 comprising input devices such as a keyboard 202, a mouse pointer device 203, a scanner 226, an external hard drive 227, and a microphone 280; and output devices including a printer 215, a display device 214 and loudspeakers 217. In some embodiments video display 214 may comprise a touchscreen.

A Modulator-Demodulator (Modem) transceiver device 216 may be used by the computer module 201 for communicating to and from a communications network 220 via a connection 221. The network 220 may be a wide-area network (WAN), such as the Internet, a cellular telecommunications network, or a private WAN. Through the network 220, computer module 201 may be connected to other similar personal devices 290 or server computers 291. Where the connection 221 is a telephone line, the modem 216 may be a traditional “dial-up” modem. Alternatively, where the connection 221 is a high capacity (e.g.: cable) connection, the modem 216 may be a broadband modem. A wireless modem may also be used for wireless connection to network 220.

The computer module 201 typically includes at least one processor 205, and a memory 206 for example formed from semiconductor random access memory (RAM) and semiconductor read only memory (ROM). The module 201 also includes a number of input/output (I/O) interfaces including: an audio-video interface 207 that couples to the video display 214, loudspeakers 217 and microphone 280; an I/O interface 213 for the keyboard 202, mouse 203, scanner 226 and external hard drive 227; and an interface 208 for the external modem 216 and printer 215. In some implementations, modem 216 may be incorporated within the computer module 201, for example within the interface 208. The computer module 201 also has a local network interface 211 which, via a connection 223, permits coupling of the personal device 200 to a local computer network 222, known as a Local Area Network (LAN).

As also illustrated, the local network 222 may also couple to the wide network 220 via a connection 224, which would typically include a so-called “firewall” device or device of similar functionality. The interface 211 may be formed by an Ethernet circuit card, a Bluetooth wireless arrangement or an IEEE 802.11 wireless arrangement or other suitable interface.

The I/O interfaces 208 and 213 may afford either or both of serial and parallel connectivity, the former typically being implemented according to the Universal Serial Bus (USB) standards and having corresponding USB connectors (not illustrated).

Storage devices 209 are provided and typically include a hard disk drive (HDD) 210. Other storage devices such as, an external HD 227, a disk drive (not shown) and a magnetic tape drive (not shown) may also be used. An optical disk drive 212 is typically provided to act as a non-volatile source of data. Portable memory devices, such as optical disks (e.g.: CD-ROM, DVD, Blu-Ray Disc), USB-RAM, external hard drives and floppy disks for example, may be used as appropriate sources of data to the personal device 200. Another source of data to personal device 200 is provided by the at least one server computer 291 through network 220.

The components 205 to 213 of the computer module 201 typically communicate via an interconnected bus 204 in a manner that results in a conventional mode of operation of personal device 200. In the embodiment shown in FIGS. 2A and 2B, processor 205 is coupled to system bus 204 through connections 218. Similarly, memory 206 and optical disk drive 212 are coupled to the system bus 204 by connections 219. Examples of personal devices 200 on which the described arrangements can be practiced include IBM-PC's and compatibles, Sun Sparc stations, Apple computers; smart phones; tablet computers or a like device comprising a computer module like computer module 201. It is to be understood that when personal device 200 comprises a smart phone or a tablet computer, display device 214 may comprise a touchscreen and other input and output devices may not be included such as, mouse pointer device 203; keyboard 202; scanner 226; and printer 215.

FIG. 2B is a detailed schematic block diagram of processor 205 and a memory 234. The memory 234 represents a logical aggregation of all the memory modules, including the storage device 209 and semiconductor memory 206, which can be accessed by the computer module 201 in FIG. 2A.

The methods of the invention may be implemented using personal device 200 wherein the methods may be implemented as one or more software application programs 233 executable within computer module 201. In particular, the steps of the methods of the invention may be effected by instructions 231 in the software carried out within the computer module 201

The software instructions 231 may be formed as one or more code modules, each for performing one or more tasks. The software 233 may also be divided into two separate parts, in which a first part and the corresponding code modules performs the method of the invention and a second part and the corresponding code modules manage a graphical user interface between the first part and the user.

The software 233 may be stored in a computer readable medium, including in a storage device of a type described herein. The software is loaded into the personal device 200 from the computer readable medium or through network 221 or 223, and then executed by personal device 200. In one example the software 233 is stored on storage medium 225 that is read by optical disk drive 212. Software 233 is typically stored in the HDD 210 or the memory 206.

A computer readable medium having such software 233 or computer program recorded on it is a computer program product. The use of the computer program product in the personal device 200 preferably effects a device or apparatus for implementing the methods of the invention.

In some instances, the software application programs 233 may be supplied to the user encoded on one or more disk storage medium 225 such as a CD-ROM, DVD or Blu-Ray disc, and read via the corresponding drive 212, or alternatively may be read by the user from the networks 220 or 222. Still further, the software can also be loaded into the personal device 200 from other computer readable media. Computer readable storage media refers to any non-transitory tangible storage medium that provides recorded instructions and/or data to the computer module 201 or personal device 200 for execution and/or processing. Examples of such storage media include floppy disks, magnetic tape, CD-ROM, DVD, Blu-ray Disc, a hard disk drive, a ROM or integrated circuit, USB memory, a magneto-optical disk, or a computer readable card such as a PCMCIA card and the like, whether or not such devices are internal or external of the computer module 201. Examples of transitory or non-tangible computer readable transmission media that may also participate in the provision of software application programs 233, instructions 231 and/or data to the computer module 201 include radio or infra-red transmission channels as well as a network connection 221, 223, 334, to another computer or networked device 290, 291 and the Internet or an Intranet including email transmissions and information recorded on Websites and the like.

The second part of the application programs 233 and the corresponding code modules mentioned above may be executed to implement one or more graphical user interfaces (GUIs) to be rendered or otherwise represented upon display 214. Through manipulation of, typically, keyboard 202, mouse 203 and/or screen 214 when comprising a touchscreen, a user of personal device 200 and the methods of the invention may manipulate the interface in a functionally adaptable manner to provide controlling commands and/or input to the applications associated with the GUI(s). Other forms of functionally adaptable user interfaces may also be implemented, such as an audio interface utilizing speech prompts output via loudspeakers 217 and user voice commands input via microphone 280. The manipulations including mouse clicks, screen touches, speech prompts and/or user voice commands may be transmitted via network 220 or 222.

When the computer module 201 is initially powered up, a power-on self-test (POST) program 250 may execute. The POST program 250 is typically stored in a ROM 249 of the semiconductor memory 206. A hardware device such as the ROM 249 is sometimes referred to as firmware. The POST program 250 examines hardware within the computer module 201 to ensure proper functioning, and typically checks processor 205, memory 234 (209, 206), and a basic input-output systems software (BIOS) module 251, also typically stored in ROM 249, for correct operation. Once the POST program 250 has run successfully, BIOS 251 activates hard disk drive 210. Activation of hard disk drive 210 causes a bootstrap loader program 252 that is resident on hard disk drive 210 to execute via processor 205. This loads an operating system 253 into RAM memory 206 upon which operating system 253 commences operation. Operating system 253 is a system level application, executable by processor 205, to fulfil various high level functions, including processor management, memory management, device management, storage management, software application interface, and generic user interface.

Operating system 253 manages memory 234 (209, 206) in order to ensure that each process or application running on computer module 201 has sufficient memory in which to execute without colliding with memory allocated to another process. Furthermore, the different types of memory available in the personal device 200 must be used properly so that each process can run effectively. Accordingly, the aggregated memory 234 is not intended to illustrate how particular segments of memory are allocated, but rather to provide a general view of the memory accessible by computer module 201 and how such is used.

Processor 205 includes a number of functional modules including a control unit 239, an arithmetic logic unit (ALU) 240, and a local or internal memory 248, sometimes called a cache memory. The cache memory 248 typically includes a number of storage registers 244, 245, 246 in a register section storing data 247. One or more internal busses 241 functionally interconnect these functional modules. The processor 205 typically also has one or more interfaces 242 for communicating with external devices via the system bus 204, using a connection 218. The memory 234 is connected to the bus 204 by connection 219.

Application program 233 includes a sequence of instructions 231 that may include conditional branch and loop instructions. Program 233 may also include data 232 which is used in execution of the program 233. The instructions 231 and the data 232 are stored in memory locations 228, 229, 230 and 235, 236, 237, respectively. Depending upon the relative size of the instructions 231 and the memory locations 228-230, a particular instruction may be stored in a single memory location as depicted by the instruction shown in the memory location 230. Alternately, an instruction may be segmented into a number of parts each of which is stored in a separate memory location, as depicted by the instruction segments shown in the memory locations 228 and 229.

In general, processor 205 is given a set of instructions 243 which are executed therein. The processor 205 then waits for a subsequent input, to which processor 205 reacts by executing another set of instructions. Each input may be provided from one or more of a number of sources, including data generated by one or more of the input devices 202, 203, or 214 when comprising a touchscreen, data received from an external source across one of the networks 220, 222, data retrieved from one of the storage devices 206, 209 or data retrieved from a storage medium 225 inserted into the corresponding reader 212. The execution of a set of the instructions may in some cases result in output of data. Execution may also involve storing data or variables to the memory 234.

The disclosed arrangements use input variables 254 that are stored in the memory 234 in corresponding memory locations 255, 256, 257, 258. The described arrangements produce output variables 261 that are stored in the memory 234 in corresponding memory locations 262, 263, 264, 265. Intermediate variables 268 may be stored in memory locations 259, 260, 266 and 267.

The register section 244, 245, 246, the arithmetic logic unit (ALU) 240, and the control unit 239 of the processor 205 work together to perform sequences of micro-operations needed to perform “fetch, decode, and execute” cycles for every instruction in the instruction set making up the program 233. Each fetch, decode, and execute cycle comprises:

(a) a fetch operation, which fetches or reads an instruction 231 from memory location 228, 229, 230;

(b) a decode operation in which control unit 239 determines which instruction has been fetched; and

(c) an execute operation in which the control unit 239 and/or the ALU 240 execute the instruction.

Thereafter, a further fetch, decode, and execute cycle for the next instruction may be executed. Similarly, a store cycle may be performed by which the control unit 239 stores or writes a value to a memory location 232.

Each step or sub-process in the methods of the invention may be associated with one or more segments of the program 233, and may be performed by register section 244-246, the ALU 240, and the control unit 239 in the processor 205 working together to perform the fetch, decode, and execute cycles for every instruction in the instruction set for the noted segments of program 233.

One or more other computers 290 may be connected to the communications network 220 as seen in FIG. 2A. Each such computer 290 may have a similar configuration to the computer module 201 and corresponding peripherals.

One or more other server computer 291 may be connected to the communications network 220. These server computers 291 respond to requests from the personal device or other server computers to provide information.

Method 10 may alternatively be implemented in dedicated hardware such as one or more integrated circuits performing the functions or sub functions of the described methods. Such dedicated hardware may include graphic processors, digital signal processors, or one or more microprocessors and associated memories.

It will be understood that in order to practice the methods of the invention as described above, it is not necessary that the processors and/or the memories of the processing machine be physically located in the same geographical place. That is, each of the processors and the memories used in the invention may be located in geographically distinct locations and connected so as to communicate in any suitable manner. Additionally, it will be understood that each of the processor and/or the memory may be composed of different physical pieces of equipment. Accordingly, it is not necessary that a processor be one single piece of equipment in one location and that the memory be another single piece of equipment in another location. That is, it is contemplated that the processor may be two pieces of equipment in two different physical locations. The two distinct pieces of equipment may be connected in any suitable manner. Additionally, the memory may include two or more portions of memory in two or more physical locations.

To explain further, processing as described above is performed by various components and various memories. It will be understood, however, that the processing performed by two distinct components as described above may, in accordance with a further embodiment of the invention be performed by a single component. Further, the processing performed by one distinct component as described above may be performed by two distinct components. In a similar manner, the memory storage performed by two distinct memory portions as described above may, in accordance with a further embodiment of the invention, be performed by a single memory portion. Further, the memory storage performed by one distinct memory portion as described above may be performed by two memory portions.

Further, various technologies may be used to provide communication between the various processors and/or memories, as well as to allow the processors and/or the memories of the invention to communicate with any other entity, i.e., so as to obtain further instructions or to access and use remote memory stores, for example. Such technologies used to provide such communication might include a network, the Internet, Intranet, Extranet, LAN, an Ethernet, a telecommunications network (e.g., a cellular or wireless network) or any client server system that provides communication, for example. Such communications technologies may use any suitable protocol such as TCP/IP, UDP, or OSI, for example.

In this specification, the terms “comprises”, “comprising” or similar terms are intended to mean a non-exclusive inclusion, such that an apparatus that comprises a list of elements does not include those elements solely, but may well include other elements not listed.

Throughout the specification the aim has been to describe the invention without limiting the invention to any one embodiment or specific collection of features. Persons skilled in the relevant art may realize variations from the specific embodiments that will nonetheless fall within the scope of the invention.

1. A system for directly tracking human activity comprising:
a plurality of interactive activity objects that interact with each other in one or more activities involving physical manipulation of the interactive activity objects by a person;
at least one wearable device associated with the person, the wearable device tracking the person's activity by sensing the person's movements, to obtain data directly relating to the person's activity; and
a database to record the person's direct activity and at least some of the person's direct physical manipulation of the interactive activity objects.

2. The system of claim 1, wherein the person is a child, and the interactive activity objects are toys that are used for active play.

3. The system of claim 2, wherein the interactive activity objects are construction toys.

4. The system of claim 3, wherein the construction toys comprise one or more of bricks or blocks having connecting components to connect the bricks or blocks together.

5. The system of claim 1, wherein the database comprises data relating to multiple persons.

6. The system of claim 1, wherein the at least one wearable device is worn about the wrist of the person.

7. The system of claim 1, wherein the at least one wearable device comprises a mobile computing device.

8. The system of claim 1, wherein the at least one wearable device comprises a display to display information regarding the person's activity.

9. The system of claim 1, wherein the at least one wearable device comprises a control interface to receive manual input regarding the person's activity with the interactive activity object.

10. The system of claim 1, wherein the database is stored on a remote server.

11. The system of claim 10, wherein the data is uploaded to the remote server via the Internet.

12. The system of claim 1, wherein the data comprises user data and activity data relating to the person.

13. The system of claim 12, wherein the user data comprises health data relating to the person, and the health data is tracked over time.

14. The system of claim 1, wherein a goal comprising one or both of an activity goal and a kit assembly goal may be achieved.

15. The system of claim 14 wherein achievement of the goal requires verification.

16. The system of claim 15 wherein the verification is provided by a parent or guardian, or by uploading a photograph of the completed assembly configuration, and/or by artificial intelligence to analyse the person's movements and/or the photographs provided and verify that they have achieved a particular goal.

17. The system of claim 12, wherein the activity data comprises at least one of:
the duration the person has performed activities;
the amount of movement performed by the person when performing activities; and/or
the amount of energy used by the person when performing activities.

18. A computer-implemented method of directly tracking human activity comprising:
obtaining data regarding a person's direct activity with a plurality of interactive activity objects that interact with each other in one or more activities involving physical manipulation of the interactive activity objects by a person, the data obtained by sensing a person's movements with at least one wearable device associated with the person, the wearable device tracking the person's activity by sensing the person's movements, to obtain data directly relating to the person's activity; and
uploading the data comprising the person's direct activity and at least some of the person's direct physical manipulation of the interactive activity objects to a database.

19. A mobile computing device for tracking interactive activity of a person, comprising:
at least one processor; and
a memory in communication with the processor, the memory storing instructions that, when executed by the at least one processor, cause the platform to perform the method of claim 18.

20. The mobile computing device of claim 19, wherein the mobile computing device comprises a mobile phone a tablet computer or a wearable device.
